(19) United States
(12) Patent Application Publication
(10) Pub. No.: US 2007/0287947 A1
(43) Pub. Date: Dec. 13, 2007

(54) Device and Method for Inhibiting Decubitus Ulcers

(76) Inventor: Murray P. Hamlet, Kingston, MA (US)

Correspondence Address: MIRICK, O'CONNELL, DEMALLIE & LOUGEE, LLP, 1700 WEST PARK DRIVE, WESTBOROUGH, MA, 01581, US

(21) Appl. No.: 11/759,012

(22) Filed: Jun. 6, 2007

Related U.S. Application Data
(60) Provisional application No. 60/804,024, filed on Jun. 6, 2006.

(51) Int. Cl. A61F 15/00

(52) U.S. Cl. 602/54

(57) ABSTRACT

A method and device to promote the healing, or prevent the breakdown, of skin that may be subject to breakdown from body weight pressure. A support pad that defines a number of bristles or blades is placed so as to support the skin and underlying tissue on the multiple small points and/or edges of the pad bristles or blades.

### CROSS-REFERENCE TO RELATED APPLICATION

This application claims priority of Provisional application Ser. No. 60/804,024, filed on Jun. 6, 2006, the disclosure of which is incorporated herein by reference.

### FIELD OF THE INVENTION

This invention relates to a device and method that inhibit decubitus ulcers.

### BACKGROUND OF THE INVENTION

Decubitus ulcers (DU) are produced by point pressure over bony prominences. As skin ages, it loses its underlying fat layer which usually distributes pressure over a wider area. The skin itself also loses fat. The diameter of the bony prominence, the weight of the patient, and the duration of the pressure are important factors in the production of DU. When the interdermal pressure exceeds the capillary filling pressure, the skin becomes ischemic and dies. See the diagram of FIG. 1, which explains the formation of DU 10. DU's are cold thermo-graphically, which is why they are slow to heal. This dead skin slowly liquefies and becomes an ideal growth medium for bacteria. Open, oozing, foul-smelling, grey lesions are the result. DU's are time-consuming to prevent and treat and are the leading cause of death in rest homes and long term care centers.

There is a well-described animal correlate available. Falconers for centuries have described a lesion on the distal end of the tarso-metatarsus [palm] of the foot the call Bumble-foot. This scab-like lesion can be removed and its under-side is a white caseus material. Birds have little fat in their skin and they lack liquefying enzymes in their white blood cells. Long term perching on hawk perches produces an ischemic ulcer called Bumble-foot. The patho-physiology is the same as DU's Current DU treatment devices compress under the weight of the patient, forming smooth large contact areas, which accounts for their failure in preventing DU's. Sheep's wool, inflatable beds, foam pads, egg-crate pads and the like all fail to prevent skin ischemia. See FIG. 2 for a diagram that shows how a “pressure dressing” 12, such as those described just above, simply enlarges the weight-distribution area, which does not alleviate the problem, thus still allowing the formation of DU 10a. Failure of these products is evident in the anatomy of the skin lesions. The skin dies at a central point and there are concentric rings of dying skin from the central point outward. The rings indicate the decreasing pressure from the center outward to viable skin. Inflatable knobs and foam knobs compress to form a large diameter constant pressure point and the bed-sheets make it worse.

DU's remain a major cost of care and a significant mortality factor. There is a pressing need for an effective means to help prevent the formation of DU's, and help in their treatment.

### SUMMARY OF THE INVENTION

For skin to remain viable the pressure of contact must be less than the capillary filling pressure of a given area of skin. The contact area must be small enough to allow perfusion of tissue around the compressed area. By supporting the weight on sharp points and edges there is sufficient perfusion to allow nutrition of tissue between the points.

Prevention/treatment of DU's involves changing the biophysics of the DU production process. The weight of the patient must be distributed over a wider area, and the skin needs to be supported on points and not on smooth surfaces. Point support allows for the nutritive capillary flow between the points so the skin does not become ischemic.

This invention features a method and device to promote the healing, or prevent the breakdown, of skin that may be subject to breakdown from body weight pressure, comprising supporting the skin and underlying tissue on multiple small points and/or edges of a support pad that defines a number of resilient bristles or blades. The support pad may include an adhesive material that removably attaches the pad to the skin. The pad may be made of plastic material. The bristles or blades may be shaped so that the multiple small points and/or edges of tissue contact are provided by the end-points of the sides of straight or curvilinear bristles or blades projecting from a base. The projections may be arranged in a manner that provides multiple skin contact points per square inch. The multiple small points of tissue contact may be arranged to support the tissue off the base of the pad while allowing blood circulation to the tissue between and proximate to the multiple points of skin contact. The blood circulation to the tissue is sufficient to support the maintenance or re-establishment of skin.

The pad may be disposable. The pad may be packaged to meet standards of medical sterility. The pad may be non-disposable. There may further be included a material cleaning system that adequately prevents the non-disposable material from becoming a source of tissue infection. The material cleaning system may utilize an anti-microbial cleaning solution, or may utilize a high-pressure washing system.

The pad may be silver-impregnated. The adhesive may be air-porous and impervious to liquids and bacteria. The adhesive may be in contact with the pad bristles. The pad may have a backing and the adhesive may be in contact with the backing. The pad may have a backing that is occlusive, or that is perforated or that is constructed to be non-skid, to help maintain the pad in place. The bristles or blades may be long enough to create a space between the skin and the pad backing to collect body fluid away from the skin. The bristles or blades may be of different lengths and/or different stiffness and curvature to allow for varying levels of body weight support in order to accommodate varying body weights and size of patient pressure points, and varying levels of comfort in sensient patients.

### BRIEF DESCRIPTION OF THE DRAWINGS

Other objects, features and advantages will occur to those skilled in the art from the following detailed description of the invention, and the accompanying drawings, in which:

FIG. 1 is a schematic cross-sectional view of a decubitus ulcer over a bony prominence in a patient;

FIG. 2 is a schematic cross-sectional view of a decubitus ulcer over a bony prominence in a patient, with the use of a prior art pressure dressing;

FIG. 3 is a schematic cross-sectional view showing an embodiment of the method and construction of this invention for treating a DU, in which the skin over the bony prominence is supported in a manner that re-establishes blood flow and supports the growth of capillaries overlying the prominence;

FIG. 4 is a schematic cross-sectional view of the location shown in FIG. 3 as the DU continues to heal; and

FIG. 5 is a schematic cross-sectional view showing another embodiment of the method and construction of this invention, for preventing the formation of DU's over a bony prominence, in which the skin over the prominence is supported in a manner that allows for capillary blood flow and thus supports the maintenance of healthy tissue overlying the prominence.

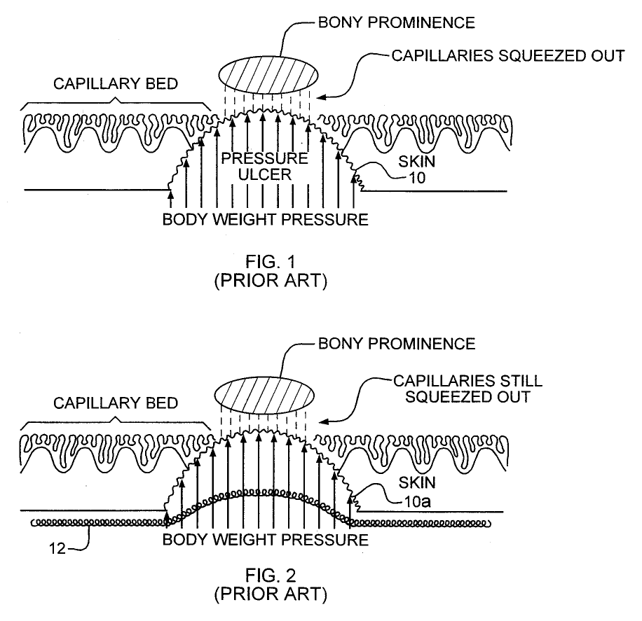

### DETAILED DESCRIPTION OF THE INVENTION

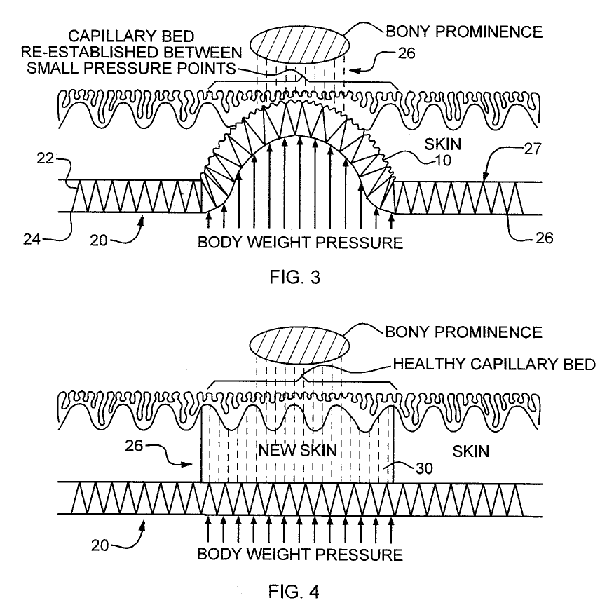

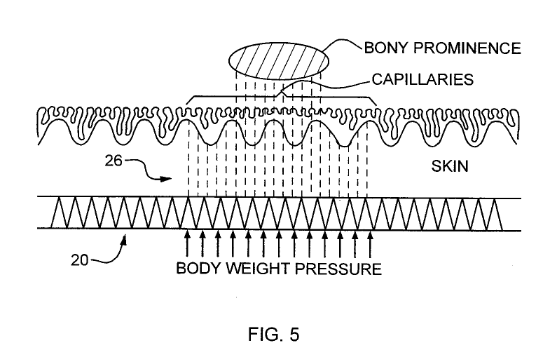

The invention is accomplished by providing a pad 20, FIGS. 3-5, that is located at patient pressure points, such as the seat, elbows, heels and back; the particular areas depending on the patient and the manner in which the patient is supported, such as in a bed or a wheelchair, for example. Pad 20 defines a multi-point surface defined by structures (typically bristles or blades that have some resiliency) 24 having points/edges 22, similar to a brush. Pad 20 maintains skin capillary flow. Bristles or blades 24 of pad 20 need to be resilient enough to support the patient's weight over the particular contact area, and long enough to allow for some sinking-in (bending of bristles/blades 24) without reaching the backing 26. In other words, the bristles/blades must present a contact area that is small enough to allow perfusion of tissue 26 around the compressed area, to support reestablishment of tissue 30, FIG. 4, in place of DU 10. By supporting the weight on sharp points and edges there is sufficient perfusion to allow nutritive capillary flow between the points so the skin does not become ischemic.

The product “Astroturf”™ from Solutia, Inc. has been determined by the inventor herein to meet these criteria. Astroturf was first described in U.S. Pat. No. 3,332,828. Astroturf is made from polyethylene in the form of eight, one-sixteenth by three-quarter inch kinked bristles/blades in a tuft. The tufts are arranged in parallel rows to form a bristle-like surface. There are about 40 bristles/blades per square inch.

Other brush-like surfaces could be used to fit specific needs. The size, length, thickness and packing density of the bristles/blades, and the material from which they are made, can be designed to achieve a certain weight support per area. For example, heel cups to support heel pressure while a person is seated or lying down can be relatively soft, while coccyx pads used while a person is seated in a wheelchair need to be larger and stiffer in order to support the torso weight without collapsing and losing their effectiveness. Anti-bacterial and/or wound-healing materials such as silver could be added to the bristles/blades. This could help to minimize skin and/or ulcer infection, or inhibit bacterial growth in wound exudate that collects in the pad. Different bristle/blade densities and resilience could be made for different compressive requirements.

The invention contemplates single use pads or the re-use of pads through cleaning such as with anti-microbial cleaning solution and/or a high-pressure washing system or sterilization by known means. The pads and/or the bristles or blades can be made of any material having the desired properties, such as rigid or semi-rigid material made of metal or plastic (e.g. polyethylene or Nylon) or wood or paper or other similar organic or non-organic material. The pad material can be designed to be easily cut to the desired size and shape pad.

The bristle/blade material is shaped so that the multiple small points and/or edges of tissue contact are provided by the end-points and by the sides of straight or curvilinear projections from a base. The projections are arranged in a manner that provides multiple skin contact points/edges per square inch. The multiple small points/edges of tissue contact are arranged to support the tissue off the base of the pad while allowing blood circulation to the tissue between and proximate to the multiple points of skin contact. The blood circulation to the tissue is sufficient to support the maintenance or re-establishment of skin. It is believed that a body pressure of less than about 30 mm Hg is necessary in order to support capillaries and therefore skin. The bristles/blades could be made long enough to create a space between the skin and the pad backing to collect fluid away from the skin. The bristles/blades could be of different lengths and/or different stiffness and curvature to allow for varying levels of body weight support in order to accommodate varying body weights and size of patient pressure points, and varying levels of comfort in sensient patients.

Back side 26 or the skin side 27 of pad 20 could be covered with a fluid-occlusive dressing such as Tegaderm, a 3M product that is a clear film dressing material that is adhesive, air-porous and impervious to liquids and bacteria. On front or skin side 27, this would help to keep the pad in place on the skin while keeping wound fluid in and urine, feces and other contaminants out. On back side 26 (or by designing the back side to be impervious), the dressing material can help to trap body fluid. Any such covering could be made larger than the pad to achieve skin adhesion and fluid control. Alternatively, the pad backing 26 could be perforated to allow drainage through the pad, and/or be designed to be non-skid so that it stays in place on a bed or wheelchair more easily. As yet another alternative, the pad could be attached to an adhesive material larger than the pad to keep the pad in place on the skin.

Advantages/uses of the invention:
Support patient's weight on points/edges through wide range of weights.
Point/edge support allows for maintaining tissue perfusion.
Bristles/blades are resilient enough to resist compression.
Bristles/blades allow for air circulation next to the skin keeping it dry. Urine drains away from the skin.
Can be used for both prevention and treatment of DU's.
Can be used to relieve risk of long term sitting in cars, on bleachers, boats and kayaks, at office desks, etc.
Can decrease the likelihood of ulcers in high-risk patients such as diabetics, spinal-cord injuries or dementia.

Although specific features of the invention are shown in some drawings and not others, this is for convenience only as the features may be combined in accordance with the invention. Other embodiments will occur to those skilled in the art and are within the following claims.

What is claimed is:

1. A method to promote the healing, or prevent the breakdown, of skin that may be subject to breakdown from body weight pressure, comprising supporting the skin and underlying tissue on multiple small points and/or edges of a support pad that defines a number of bristles or blades.

2. The method of claim 1 in which at least the bristles or blades are made of metal or plastic or wood or paper or other similar organic or non-organic material.

3. The method of claim 2 in which the bristles or blades are shaped so that the multiple small points and/or edges of tissue contact are provided by the end-points of the sides of straight or curvilinear bristles or blades projecting from a base.

4. The method of claim 1 in which the small points and/or edges are arranged in a manner that provides multiple skin contact points per square inch.

5. The method of claim 1 in which the multiple small points of tissue contact are arranged to support the tissue off the base of the pad while allowing blood circulation to the tissue between and proximate to the multiple points of skin contact.

6. The method of claim 5 in which the blood circulation to the tissue is sufficient to support the maintenance or re-establishment of skin.

7. The method of claim 1 using a disposable pad.

8. The method of claim 1 in which the pad is packaged to meet standards of medical sterility.

9. The method of claim 1 using a non-disposable pad.

10. The method of claim 9 in which non-disposable material is used in conjunction with a material cleaning system that adequately prevents the non-disposable material from becoming a source of tissue infection.

11. The method of claim 10 in which the material cleaning system utilizes an anti-microbial cleaning solution.

12. The method of claim 10 in which the material cleaning system utilizes a high-pressure washing system.

13. The method of claim 1 in which the pad is silver-impregnated.

14. A construction to promote the healing, or prevent the breakdown, of skin that may be subject to breakdown from body weight pressure, comprising:
a support pad that defines a number of bristles or blades that, when placed against the skin, support the skin and underlying tissue on multiple small points and/or edges of the bristles or blades; and
an adhesive material that removably attaches the pad to the skin.

15. The construction of claim 14 in which at least the bristles or blades are made of metal or plastic or wood or paper or other similar organic or non-organic material.

16. The construction of claim 15 in which the bristles or blades are shaped so that the multiple small points and/or edges of tissue contact are provided by the end-points of the sides of straight or curvilinear bristles or blades projecting from a base.

17. The construction of claim 14 in which the small points and/or edges are arranged in a manner that provides multiple skin contact points per square inch.

18. The construction of claim 14 in which the multiple small points of tissue contact are arranged to support the tissue off the base of the pad while allowing blood circulation to the tissue between and proximate to the multiple points of skin contact.

19. The construction of claim 18 in which the blood circulation to the tissue is sufficient to support the maintenance or re-establishment of skin.

20. The construction of claim 14 in which the pad is disposable.

21. The construction of claim 14 in which the pad is packaged to meet standards of medical sterility.

22. The construction of claim 14 in which the pad is non-disposable.

23. The construction of claim 22 further comprising a material cleaning system that adequately prevents the non-disposable material from becoming a source of tissue infection.

24. The construction of claim 23 in which the material cleaning the system utilizes an anti-microbial cleaning solution.

25. The construction of claim 23 in which the material cleaning system utilizes a high-pressure washing system.

26. The construction of claim 14 in which the pad is silver-impregnated.

27. The construction of claim 14 in which the adhesive is air-porous and impervious to liquids and bacteria.

28. The construction of claim 14 in which the adhesive is in contact with the pad bristles or blades.

29. The construction of claim 14 in which the pad has a backing and the adhesive is in contact with the backing.

30. The construction of claim 14 in which the pad has a backing that is occlusive.

31. The construction of claim 14 in which the pad has a backing that is perforated.

31. The construction of claim 14 in which the bristles or blades are long enough to create a space between the skin and the pad backing to collect body fluid away from the skin.

32. The construction of claim 14 in which the bristles or blades are of different lengths and/or different stiffness and curvature to allow for varying levels of body weight support in order to accommodate varying body weights and size of patient pressure points, and varying levels of comfort in sensient patients.

33. The construction of claim 14 in which the pad has a backing that is constructed to be non-skid, to help maintain the pad in place.
